(19) United States
(12) Patent Application Publication
(10) Pub. No.: US 2020/0188233 A1
(43) Pub. Date: Jun. 18, 2020

(54) TRACKING APPARATUS, SYSTEMS, AND METHODS

(71) Applicant: EASY TO USE PRODUCTS, LLC, Farr West, UT (US)

(72) Inventor: Kevin D. Taylor, Ogden, UT (US)

(73) Assignee: EASY TO USE PRODUCTS, LLC, Farr West, UT (US)

(21) Appl. No.: 16/526,955

(22) Filed: Jul. 30, 2019

Related U.S. Application Data
(60) Provisional application No. 62/780,852, filed on Dec. 17, 2018.

(51) Int. Cl. A61J 7/04; G16H 10/60; G16H 20/13; G06Q 10/08

(52) U.S. Cl. CPC A61J 7/0409; G16H 10/60; G09F 3/0288; G06Q 10/087; G16H 20/13

(57) ABSTRACT

A dosage tracking device, systems and methods for tracking administration of medication can include a connector on one end to removably attach the device to a medication container. The medication container can be an original medication container including prescription and dosage information as well as the original child-proof lid. The tracking device includes a pill tracking tag connected to the connector. The tab includes manually pressable and resettable buttons disposed on the tag for tracking the administration of medication. Each button includes a button head with an upward extending protrusion and un underside extending protrusion. The button heads further include a conical portion extending from a periphery of the button heads to a rigid hold or the tab providing support for the buttons to extend through the tab. The buttons are manually pressed in and reset as individual doses of medication are taken.

### CROSS-REFERENCE TO RELATED APPLICATIONS

This patent application claims priority to U.S. Provisional Patent Application No. 62/780,852 filed Dec. 17, 2018, the contents of which are hereby incorporated herein for all purposes.

### BACKGROUND OF THE INVENTION

### 1. The Field of the Invention

The present invention relates to improved systems and apparatus for tracking administration of ingestible pills, liquid medication, and tablets.

### 2. The Relevant Technology

Patients are often required to self-administer medication according to a prescribed schedule. Adherence to, or compliance with, a medication regimen is generally defined as the extent to which patients take medications as prescribed by their health care providers. Self-admiration of such medication generally occurs over a period of time according to a prescribed dosage and schedule. Lack of adherence to a prescription is one of the leading problems with self-administration of medication today. According to the World Health Organization, medication adherence can have a more direct impact on patient outcomes than the specific treatment itself. Medication adherence can affect quality and length of life, health outcomes, and overall healthcare costs. Similar to medication, supplements often require periodic administration of tablets and pills defined by a predetermined schedule.

Since many medications, especially powerful drugs for pain, insomnia, depression and other purposes, require an accurate administration of the prescribed dosage at predetermined times missing a prescribed dose, or accidentally taking an additional dose potentially causing an over dose, can have severe consequences. It is extremely important that the person, such as a patient, keep an accurate record of the doses taken so that over administration, or under administration, of the prescribed medication does not occur. It is also important that dosages not be omitted such that continuous administration of the appropriate dose and the desired treatment is maintained.

One problem of maintaining a properly timed ingestion of the accurately prescribed dosage of medicine is of particular concern with elderly people or other people whose judgement and memory may have a tendency to forget when, which, and whether a dose of medication has been taken. This problem can also be increased when the medication itself may cause cognitive limitations. And, this problem is even more prevalent when the patient requires taking more than one dose of medicine on a daily basis often in the form of multiple daily pills or tablets.

Pharmacists normally instruct patients on the proper way to take their medications, but often the patient will forget the directions or not understand them adequately. For this reason, the label on the bottle of medication generally reiterates the dosage and administration regimen. Nevertheless, patients often forget whether they took their medication as prescribed or at the prescribed times. This problem exists for all forms of medication, including pills capsules, tablets, liquids, and other solids. The problem also applies to caretakers as well as veterinary medication prescribed for treatment of animals.

Traditionally, medication boxes have been used with individual compartments for the days of the week. Some designs may have multiple dose compartments for each day, for doses up to four times a day, for example. While such medication boxes are indicative of whether a dose for a particular day or dosing interval has been taken, they are also inconvenient to the patient since they are often bulky and must be carried around separately from the patient's medication. Also, the patient must remember to distribute the doses of medication into the appropriate boxes each week prior to the time that the dose must be taken. Other approaches can include a variety of reminders and organizers. Reminders might include a timer or alarm that alerts a person to take a pill.

Such conventional pill organizers and reminders are not directly associated with the original medication container, however. The medication bottle itself includes important information about the medication and prescription including its dosage, schedule, administration instructions, warnings, the prescribing doctor, pharmacy, manufacturer, potentially appearance of the pill, and other useful information. This information is not otherwise readily available once the medication is removed from its original package, such as a medication bottle, and placed in the compartments of the traditional medication box often intermixed with other prescribed medication and/or supplements.

Another disadvantage of traditional medication boxes and other solutions is that they no longer retain the child-proof protection afforded by the originally provided medication bottle and packaging. As previously mentioned, a traditional daily medication box, for example, will not prevent a child from accessing the potentially powerful medication held therein. Moreover, a child may mistake the pills for candy or other brightly colored food or candy thereby easily accessing more than one compartment of a readily available medication box.

Other products on the market include electronic readers or monitors which are wirelessly connected to your phone or have an LCD screen that is mounted to the container which have multiple compartments and lids with markings which indicate the day of the week. To have a container which allows multiple doses per day, and seven days in a week, becomes very large and cumbersome. And for a single prescription bottle such conventional designs are cumbersome and extreme overkill as to a more simplified and intuitive approach.

Another disadvantage of this type of container is that once a pill has been loaded in the container, the pill becomes generic or untrackable, because the warnings and special instructions are no longer kept with the pill as previously discussed. It is very difficult sometimes to tell one pill from another, and can be very dangerous if a reaction or accidental ingestion occurs. Also, most of the generic and/or daily dose containers do not have safety or child-resistant caps.

Another problem with traditional alarm and reminder systems is that it is not always set up or programmed correctly by the patient. As previously discussed, the patient may be particularly prone to forget their medication. Similarly, the same patient may be particularly prone to set up a reliable reminder or alarm system.

Similarly some conventional reminder systems suffer from a shortcoming in that it is not readily apparent that a previous administration of medication has been missed. That is, should a previous administration of medication be missed for whatever reason, for example a forgotten setting or reset, it is not readily apparent that such a missed administration of medication has occurred.

The subject matter claimed herein is not limited to embodiments that solve any disadvantages or that operate only in environments such as those described above. Rather, this background is only provided to illustrate one exemplary technology area where some embodiments described herein may be practiced

### SUMMARY

This Summary is provided to introduce a selection of concepts in a simplified form that are further described below in the Detailed Description. This Summary is not intended to identify key features or essential characteristics of the claimed subject matter, nor is it intended to be used as an aid in determining the scope of the claimed subject matter.

A dosage tracking device for tracking administration of medication can include a connector on one end to removably attach the device to a medication container, a pill tracking tag connected to the connector, and manually pressable and resettable buttons disposed on the tag for tracking the administration of medication.

The connector can include an elastic loop for affixing the dosage tracking device to an original container of the medication. The container can be an originally prescribed container of medication including original administration instructions and medication description. The tag includes a rigid tag body having holes through which the depressible and resettable buttons extend, the buttons being sized for manual articulation by fingers of a human hand. The tag can have a recessed rear surface surrounded by a peripheral rim. The peripheral rim extends above the recessed rear surface beyond the top of the underside of the buttons when the buttons are pressed in so as to prevent inadvertent reset of the buttons when placed upon a surface. A height of the peripheral rim is equal to or greater in height than a height of the buttons extending above the rear surface when the buttons are in the depressed position.

The buttons and connector can be integrally formed of an elastic silicone material and the tab is formed by two interconnected hard plastic material. The buttons are assigned to a medication administration schedule such that the user can verify the administration of medication according to the depressed buttons thereon. The buttons can include visual and/or tactile indicia associated with the medication administration schedule.

The connector can be formed integral with the buttons. The tab can comprise a two piece snap-fit construction to encapsulate the button portion, the buttons extending through holes of the assembled plastic portions and the connector extending from an end of the plastic portions.

Each button includes a button head including an upper protrusion and an underside protrusion. The button head is connected to a tapered conical portion. The tapered conical portion extends from a periphery of the button head outwardly to a supporting hole in the tab, the tab supporting the conical portion in-place. The conical portion allows for the button head to be pressed into the tab and allows for the button head to snap into the pressed-in position. As such, the buttons are allowed to be popped into the pressed-in state and then from the underside of the tab the buttons are allowed to be reset back into their reset original popped out state.

An underside protrusion of each button head extends from the underside of the tab in the pressed-in state and is manually articulable by a human finger so as to compress the conical portion and allow the button head to snap back into the original reset position. The upper protrusion of the button head can be between 4 and 8 millimeters in diameter, between 5 and 6 millimeters in diameter, for example about 5.6 millimeters in diameter. In one embodiment, the height of the button above the tab can extend between 4 and 7 millimeters, for example about 5.26 millimeters.

The connector can be an elastic silicone loop and can have a diameter of between about 20 and 40 millimeters, for example about 24 millimeters, and a thickness between about 0.5 and 2 millimeters, for example about 1 millimeter. Holes in the tab supporting the buttons are between 9 and 14 millimeters in diameter. The device can be devoid of electronic components so as to render it reusable without the need for batteries or electricity in some embodiments.

A manually articulable button can include a rigid circular support extending around and holding a periphery of the button. A button head portion has an upwardly extending protrusion and an underside extending protrusion. The button includes a conically tapered portion connecting to the button head at an inner diameter and held at an outer diameter by the rigid circular support, wherein the upwardly extending protrusion is disposed for pressing the button head into the conically tapered portion so as to press the button into a popped-in position and the underside protrusion extends from the conically tapered portion so as to press the button back into a popped out reset position.

A method for manufacturing a tracking device for tracking administration of medication can include molding a substantially flexible and elastic portion including a connector on one end and a plurality of buttons on a second end. The method can further include molding a substantially rigid tag and enclosing a portion of the substantially flexible and elastic portion having the plurality of buttons within the rigid tag. The connector of the substantially flexible and elastic portion extending beyond an end of the substantially rigid tag the tag including button holes extending through the rigid tag allowing for the buttons to extend and be depressed.

Additional features and advantages of the invention will be set forth in the description which follows, and in part will be obvious from the description, or may be learned by the practice of the invention. The features and advantages of the invention may be realized and obtained by means of the instruments and combinations particularly pointed out in the appended claims. These and other features of the present invention will become more fully apparent from the following description and appended claims, or may be learned by the practice of the invention as set forth hereinafter.

### BRIEF DESCRIPTION OF THE DRAWINGS

To further clarify the above and other advantages and features of the present invention, a more particular description of the invention will be rendered by reference to specific embodiments thereof which are illustrated in the appended drawings. It is appreciated that these drawings depict only typical embodiments of the invention and are therefore not to be considered limiting of its scope. The invention will be described and explained with additional specificity and detail through the use of the accompanying drawings in which:

FIG. 1 illustrates a medication tracking device and illustrates such device affixed to a medication container;

FIG. 2 shows the medication tracking device from a front top perspective view and from a side view with each button being placed in an original reset position;

FIG. 3 shows the medication tracking device from a rear top perspective view and side view where all of the buttons have been pressed in;

FIG. 4 shows the medication tracking device from the front with a human finger pressing one of the buttons in, a view from the rear and a second view from the rear of the tracking device showing a human finger resetting one of the buttons;

FIG. 5 shows two cross-sectional views of the medication tracking device and illustrating the portions of the innovative button structure thereof;

FIG. 6 shows the disassembled top and bottom views of the parts of the medication tracking device prior to assembly;

FIG. 7 shows the assembled parts of the medication tracking device and side view of the button structure along with examples of various dimensions according to certain embodiments thereof; and

FIG. 8 illustrates additional layouts and forms of other medication tracking devices.

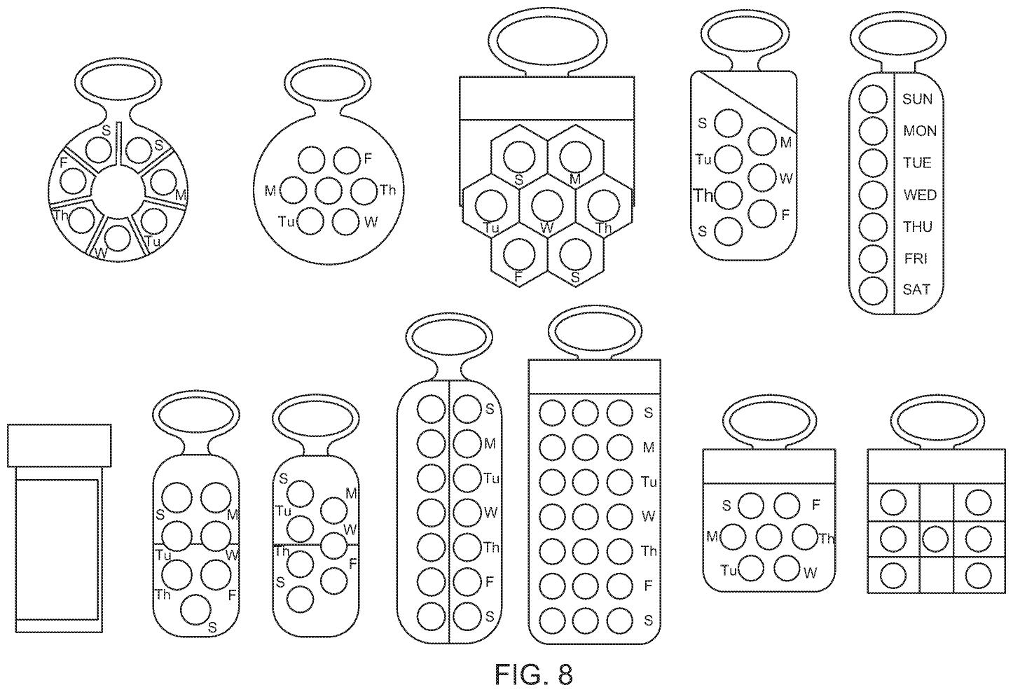

### DETAILED DESCRIPTION

Embodiments of the invention relate to medication tracking systems, apparatus, methods for manufacture thereof, and methods of use. Embodiments of the invention also relate to a unique button design which is easily depressible (“popped in”) and easily resettable (“popped out”) using certain functional features particularly articulable by a human finger or thumb. Other additional unique features, embodiments and uses thereof are disclosed hereinafter with reference to the figures.

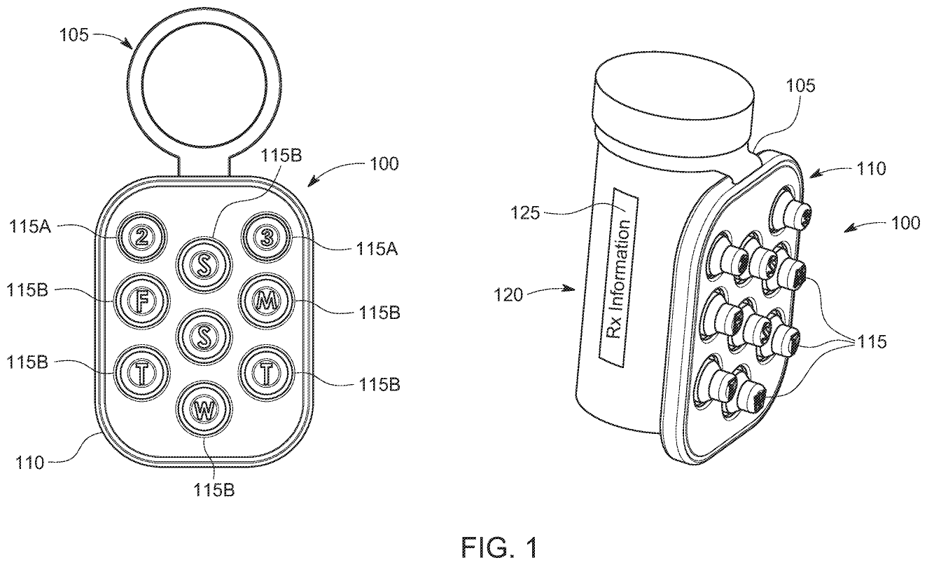

Referring to FIGS. 1, 2, and 3 a tracking device 100 is shown according to an embodiment of the invention. The tracking device 100 includes means for connecting the tracking device 100 to a bottle 120. The means for connecting the tracking device 100 can include a substantially circular shaped elastic loop 105 for holding the tracking device 100 to the bottle 120. The bottle 120 can be a prescription bottle or bottle of supplements, for example. The flexible loop 105 can be sized and shaped for a particular size of prescription bottle. The flexible loop 105 may also be substantially flexible to be highly stretchable in some embodiments so as to accommodate a wide range of bottle diameters of several times the loop's 100 original unstretched diameter.

For example, the tracking device 100 shown in FIG. 1 can be designed to help a patient keep track of the administration of medication according to the associated prescription set forth on the label 125 of the bottle. As shown in FIG. 1, the tracking device 100 can be attached to the bottle 120 of medication by stretching the loop 105 around an outer periphery of the bottle 120. In this manner the medicine may continue to be held and stored within the original prescription bottle 120 along with the original prescription instructions 125 placed on the bottle 120 by the pharmacy. These prescription instructions 125 can include the time and quantity of pills to be taken each day, for example.

Because the tracking device 100 is kept with the original bottle 120, or other original container of medication, the original child-proof safety measures of the original bottle 120 are also retained. As previously discussed, traditional medication tracking containers require the medication to be removed from the original container and do not implement sufficiently secure child proof measures to prevent accidental access to the medication by children. As shown in FIG. 1 the original medication bottle and child-proof cap are used so as to prevent such inadvertent access by children.

In addition, the tracking device 100 may be used with medications that cannot easily be removed from their original container. For example, the tracking device may be connected to the original bottle of liquids, sprays, drops, creams and other applied or ingested substances. Traditional tracking boxes and other traditional devices are not built to accommodate such substances.

According to several preferred embodiments disclosed herein, batteries and electronics can be excluded. Therefore, the tracking device 100 is truly reusable. Furthermore, the tracking device 100 can have particular advantages in low income areas in the United States and around the world to help with adherence for low income people in need of a reliable solution that does not require batteries, recharging, or even electricity. The lack of electrical reliance also makes the tracking device 100 more reliable and has a reduction of parts and reduced likelihood of failure.

The tracking device 100 includes multiple depressible and resettable buttons 115. The buttons can be pressed in (e.g. “popped in”) when a dose has been administered. Upon subsequent review of the tracking device 100 the pressed-in buttons will indicate that a particular dosage has been administered. Once a regimen of dosage administration has been completed, the pressed-in buttons 115 can be reset by pressing on an under side of the pressed-in buttons so as to reset (e.g. “pop out”) the buttons 115.

The position of the buttons can also be felt in addition to visually noticed. Therefore, someone with a lower ability to see may be able to feel the position of the buttons so as to tactically feel the position of the various button with reference to the loop indicating a top thereof.

The buttons 115 can include indicia for tracking multiple individual doses of medication for administration. The indicia can be visual, tactile, or a combination thereof. The indicia on the buttons 115 can be raised and can have a letter, number or other descriptive indication of the relevance of the button for tracking the administration of medication according to the day of the week and/or time of day, for example.

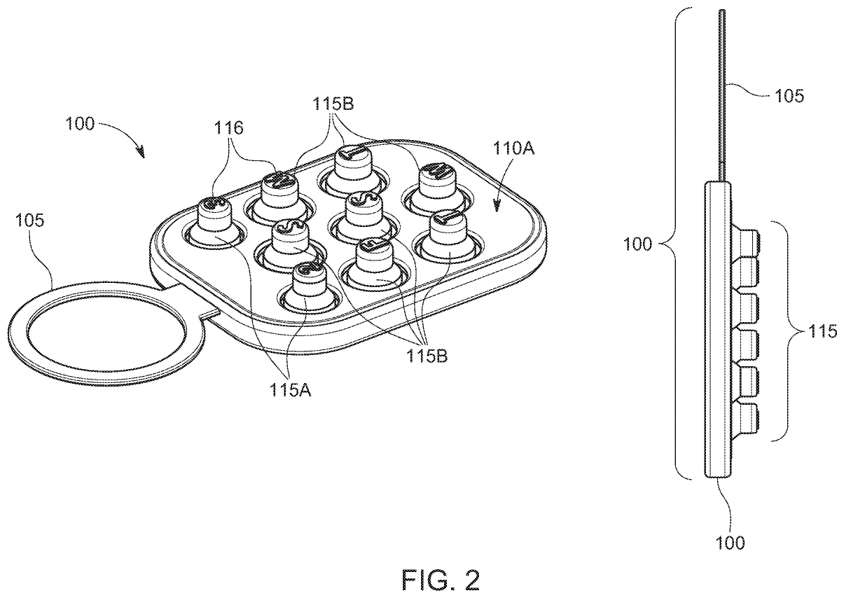

Referring to FIGS. 1 and 2, the tracking device 100 includes a first set of buttons 115A for tracking the number of the dose in a day, and a second set of buttons 115B for tracking each day of a week. As shown, the first set of buttons have a “2” and a “3” indicia in this embodiment for tracking a second and third dose of medication, the first dose indicated when neither the “2” or “3” buttons are pressed in. The second set of buttons 115B indicate the day of the week. Thus, a first of the second set of buttons 115B has an “M” associated with Monday, a second of the second set of buttons 115B has a “T” associated with Tuesday, a third of the second set of buttons 115B has a “W” associated with Wednesday, and so forth.

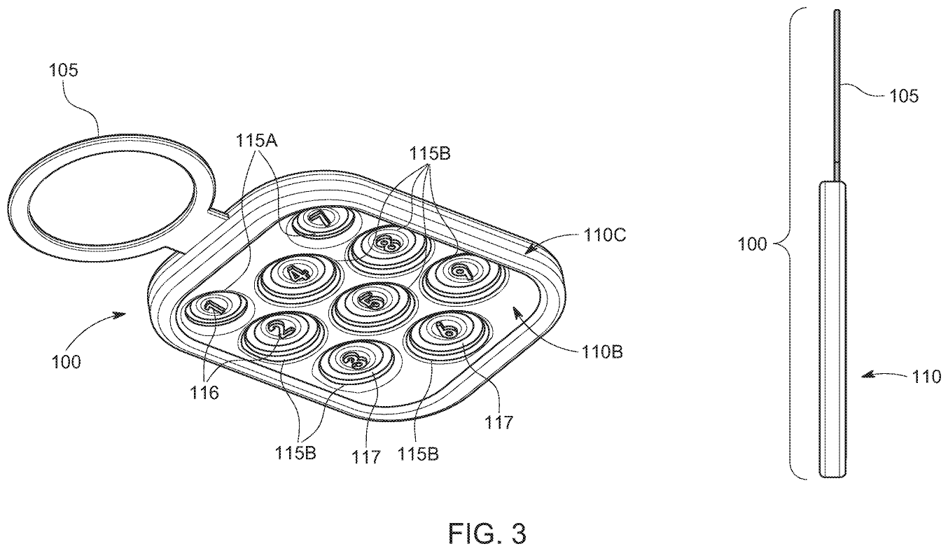

FIG. 3 shows the tracking device 100 from a rear perspective view and a side view having the buttons 115 press “popped” in. As shown in the side view of FIG. 3, the underside 110B of the tab 110 is recessed from an outer peripheral lip 110C of the tab 110 such that the buttons do not extend beyond the outer peripheral lip 110C of the tab 110 when pressed in as shown from the side view of the tracking device 100 in FIG. 3.

According to the teachings of certain embodiments disclosed herein, it is important that the underside of the buttons 115 do not extend beyond the outer peripheral lip 110C such that when placed upon a flat surface, such as on the surface of a table, the underside of the buttons 115 are not inadvertently reset. In addition to the buttons not being inadvertently reset, the recessed surface 110B of the tab 110 relative to the outer peripheral lip 110C further allows for more easy access to the underside of the buttons 115 for the tip of a person's finger to reset the buttons 115. According to certain embodiments, the depth of the recessed inner surface 110B can extend about 0.7 millimeters below the upper surface of the outer peripheral rim 110C so as to accommodate the underside protrusions 117 of the buttons 115. For example, according to such embodiments, the protrusions 117 of the buttons 115 extend approximately 0.4 millimeters above the inner surface 110B allowing for approximately 0.3 millimeters of space between the top of the underside protrusion 117 and the upper surface of the outer peripheral rim 110C in this example.

To allow for more easy access to reset the buttons 115, with particular design for the size of a human finger, the buttons 115 can include an underside articulable protrusion 116 extending therefrom from the center of the underside of the buttons 115. The underside of the buttons 115 can include indicia 116, in this example simply numbering the underside of the buttons 115. The indicia 116 can include raised numbers and letters as shown allowing for manual manipulation by the human fingertip.

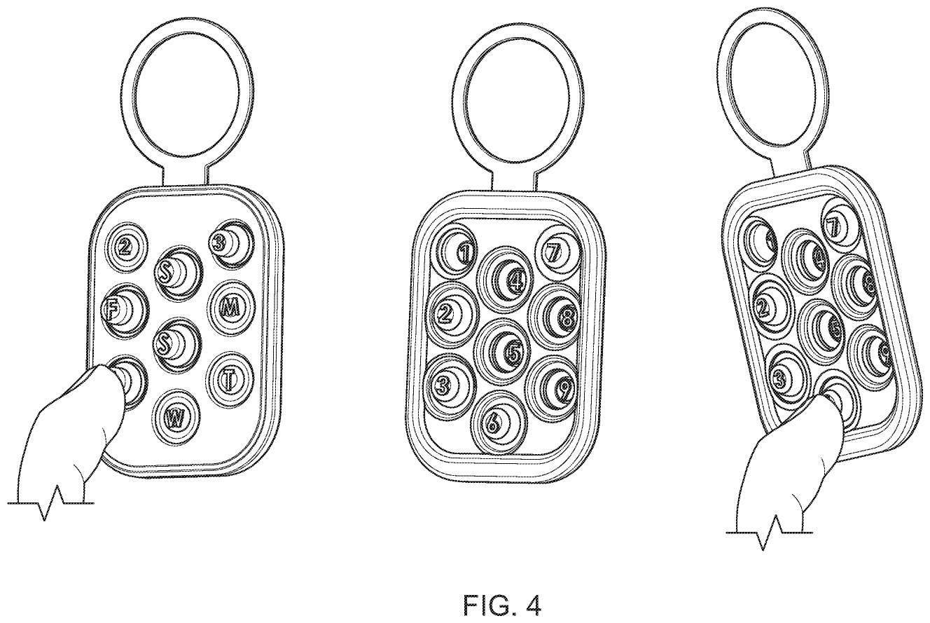

FIG. 4 illustrates the use of tracking device 100, wherein a finger is used to press-in “pop in” the first button labeled “2” of the first set of buttons 115A indicating that the second dose of Wednesday has been taken. As shown, the first three buttons of the second set of buttons 115B have been pressed in indicating that the dosage prescribed for Monday, Tuesday, and Wednesday have been taken. FIG. 4 also illustrates the back side of the tracking device 100 having the same buttons pressed in. As shown, each button has an underside protrusion extending therefrom for articulation by a human finger as shown in FIG. 4 so as to reset each button using a finger pressing against such underside as illustrated.

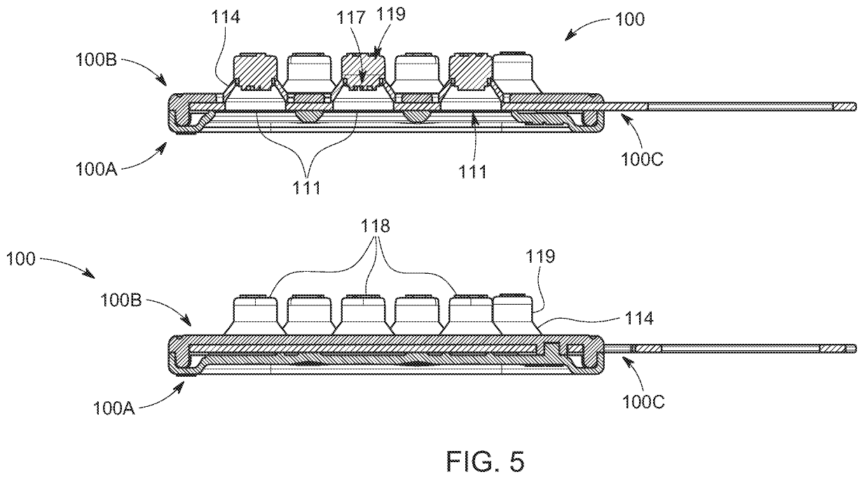

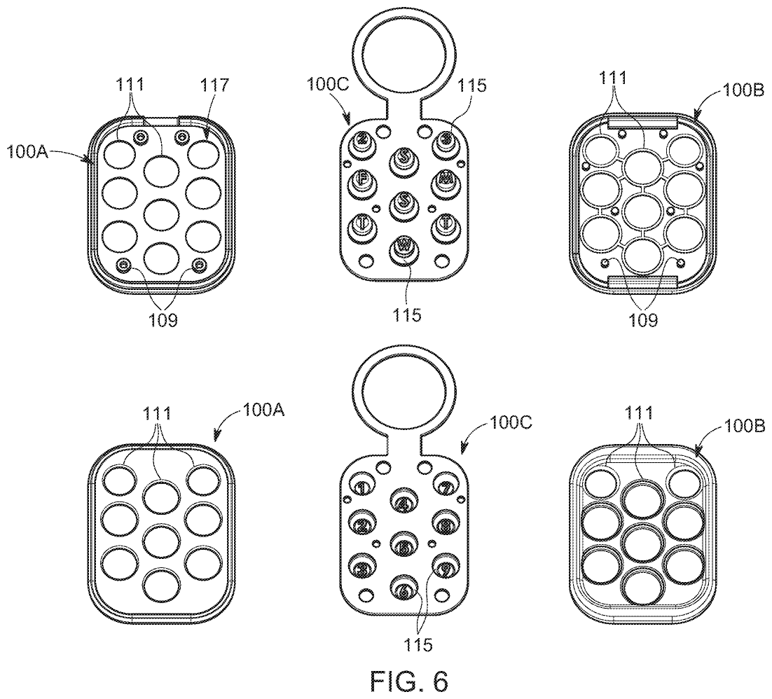

FIG. 5 illustrates cross sectional views of the tracking device 100 with particular emphasis on the assembly thereof. FIG. 6 shows the disassembled parts of the tracking device 100. With cross-reference to FIGS. 5 and 6, the tracking device 100 includes two injected molded plastic shell parts 100A and 100B and a third compression molded silicone part 100C. The plastic parts 100A and 100B lend rigidity to the tab portion 110 of the tracking device 100. The silicone part 100C provides flexibility and elasticity to the buttons 115 and loop 105 of the tracking device. Thus, the silicone part 100C can be manufactured as a single integral silicone part without the use of adhesives, seams, or multiple different manufacture processes according to this example embodiment.

The plastic parts 100A and 100B can have recessed holes 111 as shown in the disassembled illustrations of FIG. 6 to accommodate and allow access to the buttons 115 so as to manipulate the button into the different positions through the recessed holes 111. The underside 110A of the tab 110 can be recessed as shown from the outer peripheral rim 110C so as to accommodate the pressed in buttons (e.g. see FIG. 3). The plastic parts 100A and 100B can be snap-fit together using peg and hole connections and the silicone part 100C can have holes located and disposed there through and aligned with the locations of the holes and pegs of the plastic parts for easy assembly thereof.

As shown in FIG. 5, each button 115 can have a head 118 including a top protrusion 119 and the bottom protrusion 117. The head 118 can be formed as the solid button head 118. The button heads 118 are connected by a conically tapering portion 114 surrounding each button head 114. The conical portions 114 are formed integral with and extend at an angle to the button heads 118 and relative to the plastic portions 100A and 100B which securely hold the relatively larger outer periphery of the conical connecting portions 114 of the buttons 118. These conically tapering connections 114 are made of the flexible and deformable silicone allowing for deformation under manual manipulation into the recessed popped-in position and back into the reset popped-out position. When manually placed into the two different positions, the flexible conical connections 114 hold the button 115 in its popped-in or popped-out reset position. And, the opposing upper side protrusion 119 and under side protrusion 117 of the button heads 118 allow for easier fingertip articulation thereof. Thus, the size and extension distance of the upper protrusion 119 and underside protrusion 117 have been specifically selected and optimized by the inventor of this patent application for its particular applications, however the teachings disclosed herein can be applied and optimized to other buttons applications.

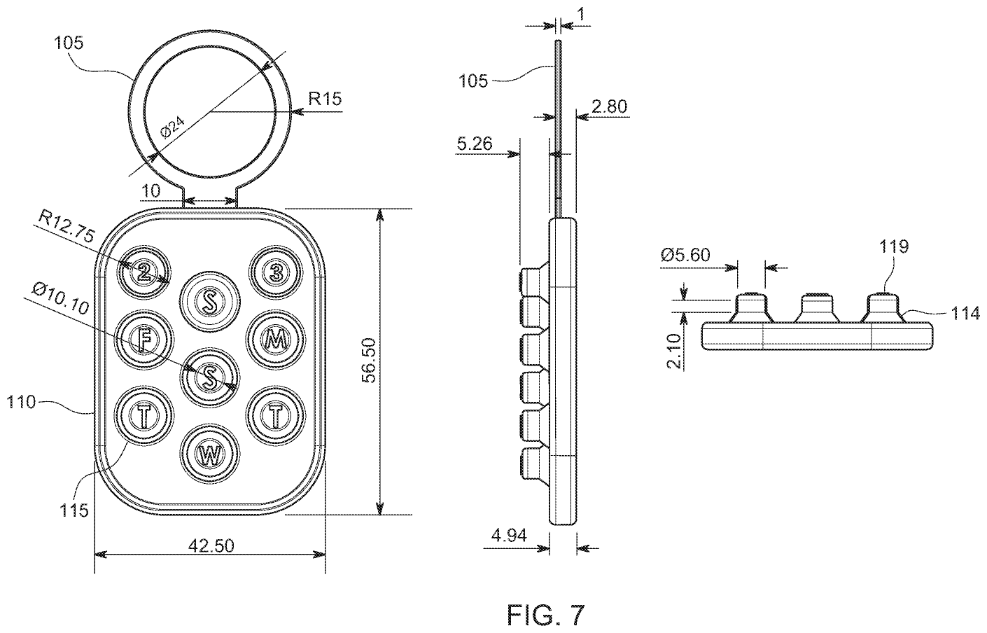

FIG. 7 illustrates some examples of dimensions in millimeters. Such dimensions can vary by 10%, 20% or more or less depending on the application. For example, a through hole dimension of 12.75 millimeters can be between about or equal to 10 and 15 millimeters and a button head diameter can be between about or equal to 4 and 7 millimeters. Similarly, an upper head height dimension can be between about 1 and 3 millimeters and a distance between a conical head upper and lower diameters can be between about 2 and 5 millimeters. The thickness of the assembled tab portion can be between 3 and 7 millimeters and the height between about 40 and 70 millimeters and width between about 30 and 60 millimeters according to some embodiments. Examples of some advantageous dimensions are disclosed herein.

Certain dimensions are illustrated therein as examples of some embodiments but should not be considered as limiting as to the invention and claims. Such dimensions, shapes and sizes may be appropriately enlarged, reduced, modified and optimized for a particular application and package design, shape and size to which the system 100 is meant to be applied.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. A dosage tracking device for tracking administration of medication comprising:
a connector on one end to removably attach the device to a medication container;
a pill tracking tag connected to the connector; and
manually pressable and resettable buttons disposed on the tag for tracking the administration of medication.

2. The device according to claim 1, the connector including an elastic loop for affixing the dosage tracking device to an original container of the medication.

3. The device according to claim 2, the container being an originally prescribed container of medication including original administration instructions and medication description.

4. The device according to claim 1, the tag including a rigid tag body having holes through which the depressible and resettable buttons extend, the buttons being sized for manual articulation by fingers of a human hand.

5. The device according to claim 1, wherein the tag has a recessed rear surface surrounded by a peripheral rim.

6. The device according to claim 5, wherein the peripheral rim extends above the recessed rear surface beyond the top of the underside of the buttons when the buttons are pressed in so as to prevent inadvertent reset of the buttons when placed upon a surface.

7. The device according to claim 5, wherein a height of the peripheral rim is equal to or greater in height than a height of the buttons extending above the rear surface when the buttons are in the depressed position.

8. The device according to claim 1, wherein the buttons and connector are integrally formed of an elastic silicone material and the tab is formed by two interconnected hard plastic material.

9. The device according to claim 1, wherein buttons are assigned to a medication administration schedule such that the user can verify the administration of medication according to the depressed buttons thereon.

10. The device according to claim 1, wherein the buttons include visual and/or tactile indicia associated with the medication administration schedule.

11. The device according to claim 1, wherein the connector is formed integral with the buttons and the tab comprises a two piece snap-fit construction to encapsulate the button portion, the buttons extending through holes of the assembled plastic portions and the connector extending from an end of the plastic portions.

12. The device according to claim 1, wherein each button includes a button head including an upper protrusion and an underside protrusion.

13. The device according to claim 12, wherein the button head is connected to a tapered conical portion.

14. The device according to claim 13, wherein the tapered conical portion extends from a periphery of the button head outwardly to a hole in the tab supporting the conical portion in-place.

15. The device according to claim 14, wherein the conical portion allows for the button head to be pressed into the tab and allows for the button head to snap into the pressed in position.

16. The device according to claim 15, wherein in the pressed-in position the underside protrusion extends from the underside of the tab and is manually articulable by a human finger so as to compress the conical portion and allow the button head to snap back into the original reset position.

17. The device according to claim 16, wherein the upper protrusion of the button head is between 4 and 8 millimeters in diameter.

18. The device according to claim 1, wherein the connector is an elastic loop and has a diameter of between about 20 and 40 millimeters and a thickness between about 0.5 and 2 millimeters.

19. The device according to claim 1, wherein holes in the tab supporting the buttons are between 9 and 14 millimeters in diameter.

20. The device according to claim 1, the device being devoid of electronic components.

21. A manually articulable button, comprising:
a rigid circular support extending around and holding a periphery of the button;
a button head portion having an upwardly extending protrusion and an underside extending protraction; and
a conically tapered portion connecting to the button head at an inner diameter and held at an outer diameter by the rigid circular support, wherein the upwardly extending protrusion is disposed for pressing the button head into the conically tapered portion so as to press the button into a popped-in position and the underside protrusion extends from the conically tapered portion so as to press the button back into a popped out reset position.

22. A method for manufacturing a tracking device for tracking administration of medication comprising:
molding a substantially flexible and elastic portion including a connector on one end and a plurality of buttons on a second end;
molding a substantially rigid tag; and
enclosing a portion of the substantially flexible and elastic portion having the plurality of buttons within the rigid tag, the connector of the substantially flexible and elastic portion extending beyond an end of the substantially rigid tag, the tag including button holes extending through the rigid tag allowing for the buttons to extend and be pressed and reset.
